(19) United States
(12) Patent Application Publication
(10) Pub. No.: US 2010/0168218 A1
(43) Pub. Date: Jul. 1, 2010

(54) METHOD OF REDUCING FOOD INTAKE

(76) Inventor: Francis P. Kuhajda, Baltimore, MD (US)

Correspondence Address: Fox Rothschild LLP, Phlla, Biotech Group, 2000 Market Street, Philadelphia, PA, 19103, US

(21) Appl. No.: 12/309,422

(22) PCT Filed: Jul. 26, 2006
(86) PCT No.: PCT/US06/28980

§ 371 Date: Nov. 19, 2009

Related U.S. Application Data
(60) Provisional application No. 60/702,323, filed on Jul. 26, 2005.

(51) Int. Cl. A61K 31/381; A61K 31/365; A61P 3/04

(52) U.S. Cl. 514/445; 514/473

(57) ABSTRACT

A method of decreasing the food intake of a subject, comprising the administration of a compound which increases FAO, where the compound does not act in the central nervous system to decrease appetite, where the compound is not a fatty acid, or an NPY-inhibitor, or an FAS inhibitor.

### BACKGROUND

The relationship between fatty acid oxidation (FAO) inhibition and increased food intake has been studied and its mechanism pursued. It has been shown in separate studies¹ that administration of C75 (trans-tetrahydro-3-methylene-2-oxo-5-n-octyl-4-furancarboxylic), a compound that both inhibits fatty acid synthase (FAS) and stimulates FAO, increases energy expenditure while reducing food intake. The decrease in food intake resulting from C75 treatment has been shown to be due reduced expression of orexigenic hypothalamic neuropeptides, such as neuropeptide-Y (NPY) leading to reduced appetite and food intake. C75 has also been shown to stimulates carnitine palmitoyltransferase-1 (CPT-1) activity leading to increased FAO (Thupari, J. N. et al., “C75 increases peripheral energy utilization and fatty acid oxidation in diet-induced obesity,” PNAS, 99: 9498-9502 (2002); Thupari, J. N., et al., “Chronic C75 Treatment of Diet-Induced Obese Mice Increases Fat Oxidation and Reduces Food Intake to Reduce Adipose Mass,” Am J Physiol Endocrinol Metab (2004). ¹Loftus, T. et al., “Reduced food intake and body weight in mice treated with fatty acid synthase inhibitors,” Science, 288, 2379-2381 (2000); Gao, S, et al., “Effect of the anorectic fatty acid synthase inhibitor C75 on neuronal activity in the hypothalamus and brainstem,” Proc Natl Acad Sci USA, 100: 5628-5633 (2003); Kim, E. K. et al., “Expression of FAS within hypothalamic neurons: a model for decreased food intake,” Am J Physiol Endocrinol Metab, 283: E867-E879 (2002)

Thus, C75 has two distinct mechanisms of action to reduce animal weight: a central anorexigenic effect in the hypothalamus (which reduces feeding), while enhancing energy expenditure peripherally (i.e. increasing FAO). However, although C75 also increased FAO and reduced food intake, its anorexigenic effect in the hypothalamus confounded the effect of FAO stimulation on food intake.

Most all of the data relating changes in FAO to food intake are based on observations of hepatic FAO inhibition. Studies from many laboratories, most notably those of Ritter and Scharrer, using a variety of pharmacological FAO inhibitors with multiple enzyme targets have demonstrated that systemic inhibition of FAO stimulates food intake in rodents. FAO inhibition increased food intake in animals fed a fat enriched diet (40% calories as fat), but was ineffective in animals consuming a low fat (7% calories as fat) diet suggesting that a dependence on fatty acid metabolism was necessary for the feeding effect. FAO inhibition shortened intermeal interval with meal size unaffected implying an effect on post-meal satiety and meal onset. Increased feeding occurred with inhibition of either CPT-1 with methyl palmoxirate, or acyl-CoA dehydrogenase with mercaptoacetate (MA), thus it was not restricted to inhibition of a single pathway enzyme and was mediated by vagal signaling.

A number of mechanisms have been proposed to link hepatic FAO inhibition to vagal activity including: depolarization of the hepatocyte membrane (16), reduction of hepatic ketone release, or more recently, by reduced hepatic energy state as measured by the ATP/AMP ratio. Information from the liver, is sent via the vagus to the nucleus of the solitary tract, projecting to the parabrachial nucleus of the pons, and then on to the central nucleus of the amygdala. Studies of c-Fos activation found additional nuclei involved including: the dorsal bed nucleus of the stria terminalis, and paraventricular nucleus (PVN) of the hypothalamus, particularly involving galanin containing neurons.

FAO inhibition clearly increases food intake via hepatic signaling through the vagus nerve. This section reviews the literature concerning altering FAO in the CNS and its effect on food intake. In brief, increasing or inhibiting FAO in the CNS had no significant effect on food intake.

Based on the work of the Kasser lab which showed that fatty acid synthesis and oxidation change in the brain in response to food intake, Beverly studied chronic icy FAO inhibition and stimulation in the ventrolateral hypothalamus (VLH) of rats. Rats were treated with infusion of icy 4-pentanoic acid (4-PA), an FAOi for 14 days, into the VLH. FAO was reduced specifically in the VLH by 37% with 4-PA, a level of reduction consistent with physiological overfeeding, but there were no significant changes in weight or carcass composition after 2 weeks of central FAO inhibition. Beverley also increased FAO in the VLH with L-carnitine infusion increasing FAO 28% over control levels consistent with physiological dietary restriction. This level of FAO stimulation did not affect food intake or animal weight. This study would support the hypothesis that changes in feeding behavior with FAO manipulation are not centrally initiated.

Langhans has presented preliminary data noting that a portal vein infusion of the medium chain fatty acid, caprylic acid in 18 h chow deprived rats, increased FAO as measured by increased plasma β-hydroxybutyrate. This maneuver reduced the size of the first dark phase meal by 38%. This abstract was not published but was part of the meeting summary material. Moreover, Langhans used a foodstuff, medium chain fatty acids, to increase FAO, not a small molecule pharmacological agent that specifically increases CPT-1 activity. (Medium chain fatty acids can bypass the CPT-1 system and directly gain access to the mitochondria for oxidation.)

Applicants have now found that increasing FAO independently results in decreased food intake.

### SUMMARY OF THE INVENTION

Accordingly, it is an object of this invention to provide a method of decreasing the food intake of a subject, comprising the administration of a compound which increases FAO, where the compound is not a fatty acid.

It is a further object of this invention to provide a method of decreasing the food intake of a subject, comprising the administration of a compound which increases FAO, where the compound does not act in the central nervous system to decrease appetite, where the compound is not a fatty acid.

It is a further object of this invention to provide a method of decreasing the food intake of a subject, comprising the administration of a compound which increases FAO, where the compound is not an NPY-inhibitor and is not a fatty acid.

It is a further object of this invention to provide a method of decreasing the food intake of a subject, comprising the administration of a compound which increases FAO, where the compound is not an FAS-inhibitor and is not a fatty acid.

### BRIEF DESCRIPTION OF THE DRAWINGS

FIG. 1 shows a scheme for synthesizing a CPT-1 stimulator.

FIG. 2 shows a scheme for synthesizing a different CPT-1 stimulator.

FIG. 3a shows the effect on weight loss of administration to pair-fed mice of a CPT-1 stimulator.

FIG. 3b shows the effect on food-intake of administration to pair-fed mice of a CPT-1 stimulator.

FIG. 3c shows the effect on fatty acid oxidation of administration to pair-fed mice of a CPT-1 stimulator.

FIG. 3d shows the effect on RER of administration to pair-fed mice of a CPT-1 stimulator.

### DETAILED DESCRIPTION OF THE INVENTION

By “FAO-stimulator,” we mean a compound which stimulates FAO as measured by oxidation of [¹⁴C]palmitate to acid soluble products in MCF7 human breast cancer cells as described by Watkins, et al., “Peroxisomal fatty acid beta-oxidation in HepG2 cells,” Arch Biochem Biophys, 289: 329-336 (1991). A compound whose Vmax is at least 125% of vehicle control is defined as an FAO stimulator.

By “NPY-inhibitor,” we mean a compound which inhibits NPY as measured by NPY mRNA using Northern blots or quantitative real-time PCR as described by Kim, et al., “Expression of FAS within hypothalamic neurons: a model for decreased food intake after C75 treatment,” Am J Physiol Endocrinol Metab, 283: E867-E879 (2002), and Kim, et al, “C75, a fatty acid synthase inhibitor, reduces food intake via hypothalamic AMP-activated protein kinase,” J Biol Chem (2004).

Increased FAO, particularly FAO in the liver leads to reduced food consumption. The mechanism of action or target of the pharmacological agent is not relevant, so long as it increases FAO in the liver without toxicity or interference with liver metabolism.

The method of the present invention may be practiced by administering compositions comprising the active ingredient(s) to humans and other animals in unit dosage forms, such as tablets, capsules, pills, powders, granules, sterile parenteral solutions or suspensions, oral solutions or suspensions, oil in water and water in oil emulsions containing suitable quantities of the compound, suppositories and in fluid suspensions or solutions. As used in this specification, the terms “pharmaceutical diluent and pharmaceutical carrier,” have the same meaning. For oral administration, either solid or fluid unit dosage forms can be prepared. For preparing solid compositions such as tablets, the compound can be mixed with conventional ingredients such as talc, magnesium stearate, dicalcium phosphate, magnesium aluminum silicate, calcium sulfate, starch, lactose, acacia, methylcellulose and functionally similar materials as pharmaceutical diluents or carriers. Capsules are prepared by mixing the compound with an inert pharmaceutical diluent and filling the mixture into a hard gelatin capsule of appropriate size. Soft gelatin capsules are prepared by machine encapsulation of a slurry of the compound with an acceptable vegetable oil, light liquid petrolatum or other inert oil.

Fluid unit dosage forms or oral administration such as syrups, elixirs, and suspensions can be prepared. The forms can be dissolved in an aqueous vehicle together with sugar, aromatic flavoring agents and preservatives to form a syrup. Suspensions can be prepared with an aqueous vehicle with the aid of a suspending agent such as acacia, tragacanth, methylcellulose and the like.

For parenteral administration fluid unit dosage forms can be prepared utilizing the compound and a sterile vehicle. In preparing solutions the compound can be dissolved in water for injection and filter sterilized before filling into a suitable vial or ampoule and sealing. Adjuvants such as a local anesthetic, preservative and buffering agents can be dissolved in the vehicle. The composition can be frozen after filling into a vial and the water removed under vacuum. The lyophilized powder can then be scaled in the vial and reconstituted prior to use.

Dose and duration of therapy will depend on a variety of factors, including (1) the patient's age, body weight, and organ function (e.g., liver and kidney function); (2) the nature and extent of the disease process to be treated, as well as any existing significant co-morbidity and concomitant medications being taken, and (3) drug-related parameters such as the route of administration, the frequency and duration of dosing necessary to effect a cure, and the therapeutic index of the drug. In general, doses will be chosen to achieve serum levels of 1 ng/ml to 100 ng/ml with the goal of attaining effective concentrations at the target site of approximately 1 μg/ml to 10 μg/ml.

### EXAMPLES

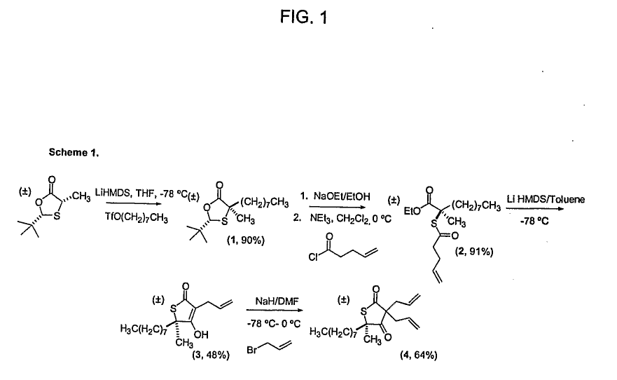

The invention will be illustrated by reference to the following examples: Compound 4 was synthesized according to the procedure outlined in FIG. 1. A synthesis of compound 4 is described in PCT Application No. US05/18443, which is incorporated herein by reference:

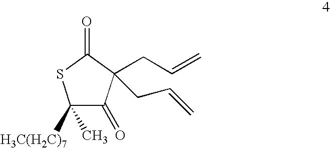

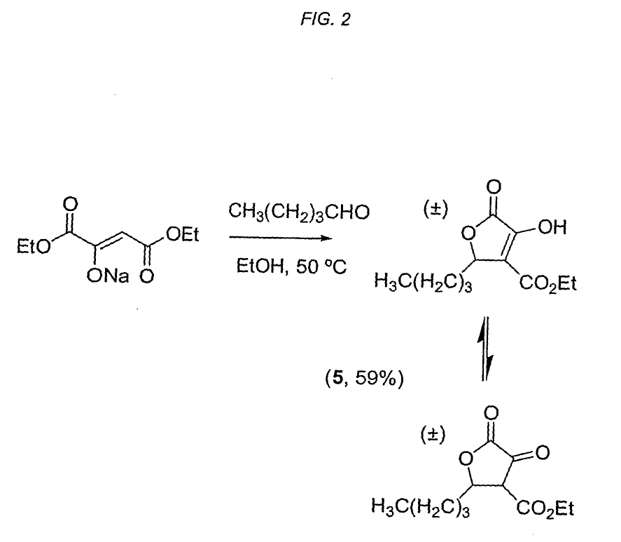

Compound 5 was synthesized according to the procedure outlined in FIG. 2. A synthesis of compound 5 is described in U.S. patent provisional application filed the same day as this application and bearing the title “NOVEL COMPOUNDS, PHARMACEUTICAL COMPOSITIONS CONTAINING SAME, AND METHODS OF USE FOR SAME:”

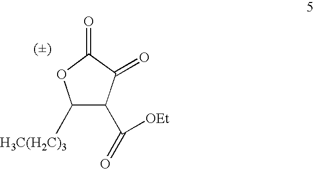

Compounds 4 and 5 were administered to pair-fed mice, and various biological properties were tested for. The results are summarized below:

TABLE 1
Compound Summary

| | | | CPT-1 | | % Maximum | Metabolic |
|---|---|---|---|---|---|---|
| | FAS Inhibition | FAO SC₁₅₀ | Activity % | % Max. Wt | Reduction in | Mechanism |
| Cpd. | IC₅₀ (μg/ml)¹ | (μg/ml)² | (μg/ml)³ | Loss⁴ | Food Intake | (indirect calorimetry) |
| 4 | Negative | 8.4 | 150 (20) | 7.9 | 68% (day 1) | ↓RER p = 0.0057, ↑VO₂ |
| 5 | Negative | 9.0 | pending | 12.3 | 50% (day 1) | ↓RER p = 0.01, ↑VO₂ |

¹“Negative” is defined as an IC₅₀ ≧100 μg/ml for slow binder, >25 μg/ml for all others. Slow binder requires preincubation at 37° C. prior to FAS assay. A test for FAS inhibition is described in PCT patent application PCT/US03/021700, the disclosure of which is hereby incorporated by reference.
²FAO SC₁₅₀ is defined as the concentration of compound (μg/ml) that yields a 50% increase in fatty acid oxidation over controls as computed by linear regression analysis. FAO is measured according to the protocol in Watkins, et al., “Peroxisomal fatty acid beta-oxidation in HepG2 cells,” Arch Biochem Biophys, 289: 329-336 (1991).
³CPT-1 was measured using digitonin permeabilized cells, as described by Thupari, et. al., “C75 increases peripheral energy utilization and fatty acid oxidation in diet-induced obesity,” PNAS, 99: 9498-9502 (2002). The number in parenthesis is concentration for maximal CPT-1 activity.
⁴Data is generated with lean female Balb/C mice with doses of 60 mg/kg, except for C75 which is 30 mg/kg.

Compound 4 does not inhibit human FAS at concentrations up to 100 μg/ml in standard and slow-binding assays. In contrast, it stimulated FAO by 150% of control at 8.4 μg/ml (28 μM) and CPT-1 activity by 150% of control at 20 μg/ml (˜60 μM). In lean female Balb/C mice, a single ip dose of 60 mg/kg caused nearly 8% weight loss within 24 hours along with a 68% reduction in food intake. Intraperitonal administration of compound 4 increased FAO as indicated by increased VO₂ compared to pair-fed animals, while reducing RER. Compound 5, with a different chemical structure, has similar biological characteristics to Compound 4.

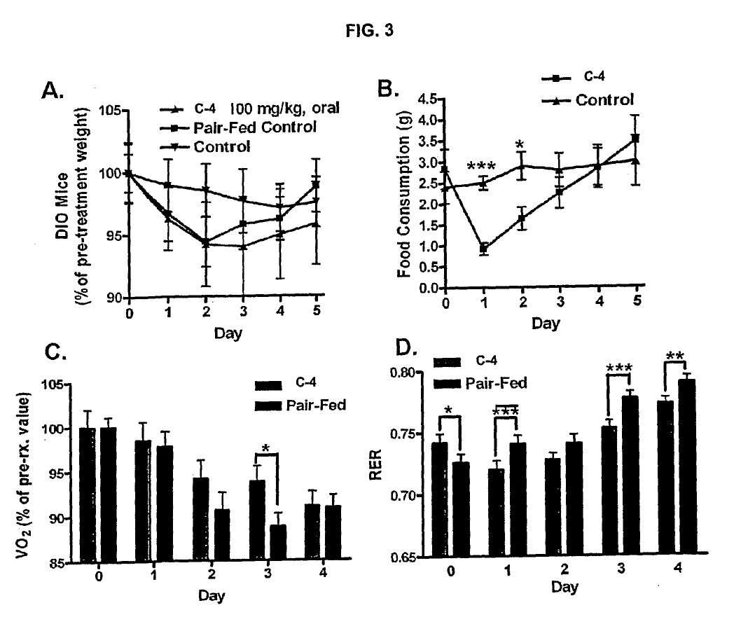

FIG. 3 expands on the data for compound 4 (C-4) presented in Table 1 above. In this experiment, compound 4 was administered orally at 100 mg/kg, in 35 μl DMSO to diet-induced obese mice, 5 animals per group. The compound 4-treated group lost more weight and maintained the weight loss longer than the pair-fed animals (FIG. 3A). The compound 4-treated animals ate significantly less food than control animals on the two days following treatment (FIG. 3B). Indirect calorimetry demonstrated that the compound 4 treated animals maintained their VO₂ compared to pair-fed animals on the first two days following treatment with a significant increase by day 3 (FIG. 3C). RER was also significantly reduced compared to the pair-fed group on days 1, 3, and 4 (FIG. 3D). Taken together, these data are consistent with increased FAO on day 1 when food intake was reduced. Similar results were obtained with ip treatment.

We claim:

1. A method of decreasing the food intake of a subject, comprising the administration of a compound which increases FAO, wherein the compound is not a fatty acid.

2. The method of claim 1, wherein the subject is an animal.

3. The method of claim 1, wherein the subject is a human.

4. The method of claim 1, wherein the compound is a CPT-1 stimulator.

5. The method of claim 1, wherein the compound is not a CPT-1 stimulator.

6. The method of claim 1, wherein the compound is a GPAT inhibitor.

7. The method of claim 1, wherein the compound is not a GPAT inhibitor.

8. A method of decreasing the food intake of a subject, comprising the administration of a compound which increases FAO, wherein the compound does not act directly in the central nervous system to decrease appetite, and further wherein the compound is not a fatty acid.

9. The method of claim 8, wherein the subject is an animal.

10. The method of claim 8, wherein the subject is a human.

11. A method of decreasing the food intake of a subject, comprising the administration of a compound which increases FAO, wherein the compound is not an NPY-inhibitor and does not reduce the expression of NPY by its direct effect in the hypothalamus, and is not a fatty acid

12. The method of claim 11 wherein the subject is an animal.

13. The method of claim 11, wherein the subject is a human.

14. A method decreasing the food intake of a subject, comprising the administration of a compound which increases FAO, wherein the compound is not an FAS-inhibitor and is not a fatty acid.

15. The method of claim 14, wherein the subject is an animal.

16. The method of claim 14, wherein the subject is a human.
